(19) United States
(12) Patent Application Publication
(10) Pub. No.: US 2025/0110192 A1
(43) Pub. Date: Apr. 3, 2025

(54) GANTRY TUBE FOR MAGNETIC RESONANCE/POSITRON EMISSION TOMOGRAPHY IMAGING SYSTEM

(71) Applicant: Siemens Medical Solutions USA, Inc., Malvern, PA (US)

(72) Inventors: Ludwig Eberler, Neumarkt i.d.OPf. (DE); James L. Corbeil, Knoxville, TN (US); Martin Schramm, Eckental (DE); Stefan Stocker, Großenseebach (DE)

(21) Appl. No.: 18/961,651

(22) Filed: Nov. 27, 2024

Related U.S. Application Data
(63) Continuation-in-part of application No. 18/040,491, filed on Feb. 3, 2023, now Pat. No. 12,189,006.
(60) Provisional application No. 63/198,285, filed on Oct. 8, 2020.

(51) Int. Cl. G01R 33/48; G01R 33/422; G01T 1/29

(52) U.S. Cl. CPC G01R 33/481; G01R 33/422; G01T 1/2985

(57) ABSTRACT

A gantry tube for a magnetic resonance/positron emission tomography imaging system. The gantry tube includes a first tube located within a second tube, wherein the first tube is oriented about a longitudinal axis of the system. The gantry tube also includes a plurality of wall elements that extend between the first and second tubes, wherein the walls and first and second tubes form a plurality of channels that extend in an axial direction substantially parallel to the longitudinal axis wherein each channel is configured to hold a PET detector of the imaging system. A PET detector is inserted into or removed from an associated channel in an axial direction from either a first end or a second end of the gantry tube.

### CROSS REFERENCE TO RELATED APPLICATION

This application is a continuation-in-part under 35 U.S.C. § 120 of copending U.S. application Ser. No. 18/040,491, filed on Feb. 3, 2023, which is a U.S. national phase application and claims priority under 35 § USC 371 of International Patent Application No. PCT/US2021/071250 filed on Aug. 23, 2021, which claims the benefit under 35 U.S.C. § 119(e) of U.S. Provisional Application No. 63/198,285, filed on Oct. 8, 2020 each of which is hereby incorporated herein by reference in its entirety.

### TECHNICAL FIELD

Aspects of the present invention relate to a gantry tube for a magnetic resonance/positron emission tomography (MR/PET) imaging system, and more particularly, to a gantry tube having a plurality of wall elements that extend between first and second tubes to form channels that extend in an axial direction substantially parallel to a longitudinal axis of the system wherein a PET detector of the system is inserted into or removed from an associated channel in the axial direction.

### BACKGROUND

A magnetic resonance/positron emission tomography (MR/PET) imaging system is a hybrid diagnostic system that includes two different imaging modalities. The integration of MR and PET imaging systems requires the location of PET detectors within a magnetic resonance imaging (MRI) system volume. Available space within an integrated MR/PET system comes at a premium of both cost and performance, with components often competing for an optimal location. For instance, a radio frequency (RF)-transmit antenna of the MRI system and the PET detectors of the PET system must both be placed as close to the patient as possible. In addition, an RF system of the MRI system and the PET electronics require electromagnetic shielding to protect each other against respective frequencies emitted by the RF system and PET detectors. As a result, a compromise solution is utilized that allows for the suitable location of the PET detectors while also allowing sufficient room for shielding and ease of access to the PET detectors during servicing or maintenance.

Referring to FIG. 1, a front view of a conventional MR/PET imaging system 10 is shown. FIG. 2 is an enlarged view of area 2 of FIG. 1. The system 10 includes a patient bore 12 or tunnel that receives a patient to be scanned, RF transmit antenna or body coil 14 (component of the MRI system), PET gantry 16 (part of the PET system) that includes a gantry tube 15, gradient coil 18 (a component of the MRI system) and superconducting magnet 20 (a component of the MRI system). The PET gantry 16 includes a plurality of PET detectors and a radio frequency (RF) screen 22 (a component of the MRI system) located on an inner surface 24 of a gantry tube 15. It would be advantageous in such systems to provide access to a single PET detector without having to disassemble or disturb substantial portions of system 10, especially if leaving the PET gantry 16 in place. The PET detectors may be located either on a separate gantry tube 15 or integrated into a RF-transmit antenna tube. In both conventional arrangements, the PET detectors are installed or loaded radially into a supporting tube. In order to gain access to the PET detectors, removal of a substantial portion of the structure from the system 10 is required even if only one PET detector needs servicing. Referring to FIG. 3, a perspective view of the system 10 is shown wherein the PET gantry 16 is in an extended position. In this position, PET detectors 26 of the system 10 are exposed thus enabling servicing of the PET detectors 26. A first end 28 of the PET gantry 16 is supported by a backplane 30 that is moveably attached to service rails 32 that enable separation of the backplane 30 from a remaining portion 34 of the system 10 and movement of the PET gantry 16 to the extended position. A second end 36 of the PET gantry 16, opposite the first end 28, stays in the remaining portion 34 and is supported by rolling elements. The gantry tube 15 is configured to hold the PET detectors 26 such that the PET detectors 26 are inserted into the gantry tube 15, or removed from the gantry tube 15 (exemplary PET detector 38 is shown in FIG. 2), in a direction substantially transverse to a longitudinal center axis 40 of the system 10 (i.e., in a radial direction) as shown by arrow 42.

### SUMMARY OF THE INVENTION

A gantry tube for a magnetic resonance/positron emission tomography (MR/PET) imaging system is disclosed wherein the imaging system includes a gradient coil located within a superconducting magnet and wherein the imaging system further includes a body coil located within a radio frequency (RF) screen and wherein the gantry tube is located between the gradient coil and RF screen. The gantry tube includes a first tube located within a second tube, wherein the first tube is oriented about a longitudinal axis of the system. An inner surface of the body coil defines a patient bore for receiving a patient to be scanned. The gantry tube also includes a plurality of wall elements that extend between the first and second tubes, wherein the walls and first and second tubes form a plurality of channels that extend in an axial direction substantially parallel to the longitudinal axis wherein each channel is configured to hold a detector of the system. A PET detector is inserted into or removed from an associated channel in the axial direction from either a first end or a second end of the gantry tube. The gantry tube is fabricated by joining a plurality of segments that each include sections of the first and second tubes, walls and a plurality of channels wherein each segment is fabricated using a nonconductive material.

In addition, a method of inserting a PET detector into a gantry tube of a magnetic resonance/positron emission tomography (MR/PET) imaging system or removing a PET detector from the gantry tube is disclosed wherein the imaging system includes a gradient coil located within a superconducting magnet and the imaging system further includes a body coil located within a radio frequency (RF) screen and wherein the gantry tube is located between the gradient coil and the RF screen. The method includes providing a first tube located within a second tube and wherein the first tube is oriented about a longitudinal axis of the system. The method also includes providing a plurality of wall elements that extend between the first and second tubes to form a plurality of channels wherein the channels extend in an axial direction substantially parallel to the longitudinal axis. In addition, the method includes joining a plurality of segments that each include sections of the first and second tubes, walls and a plurality of channels to form the gantry tube wherein each segment is fabricated using a nonconductive material. Further, the method includes moving a PET detector in the axial direction to either insert the PET detector into an associated channel or remove a PET detector from an associated channel.

Those skilled in the art may apply the respective features of the present invention jointly or severally in any combination or sub-combination.

### BRIEF DESCRIPTION OF THE DRAWINGS

The exemplary embodiments of the invention are further described in the following detailed description in conjunction with the accompanying drawings, in which:

FIG. 1 is a front view of a prior art MR/PET imaging system.

FIG. 2 is an enlarged view of area 2 of FIG. 1.

FIG. 3 is a perspective view of a prior art MR/PET imaging system wherein a PET gantry is shown in an extended position.

FIG. 4 is a front view of a gantry tube used to form a PET gantry in accordance with an aspect of the invention.

FIG. 5 depicts an enlarged view of an exemplary channel that receives a PET detector.

FIG. 6 depicts a segment of the gantry tube.

FIGS. 7A and 7B depict perspective patient and service end views of the gantry tube.

FIG. 8 is a front view of an MR/PET imaging system that includes the gantry tube of the invention.

FIG. 9 depicts an extension tube for a PET detector positioning device wherein a first end of the extension tube includes a rotatable latch device.

FIG. 10 depicts the extension tube having a second end that is attached to a PET detector to form a tube and PET detector assembly.

FIG. 11 is a perspective view of the latch device.

FIG. 12 is a partial side view of a first support ring and second tube and the tube and PET detector assembly.

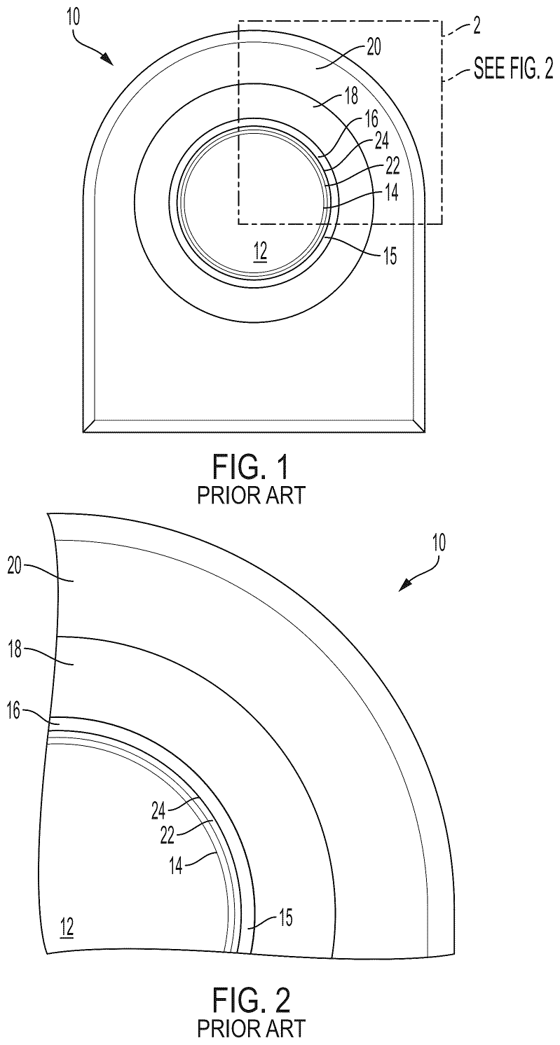

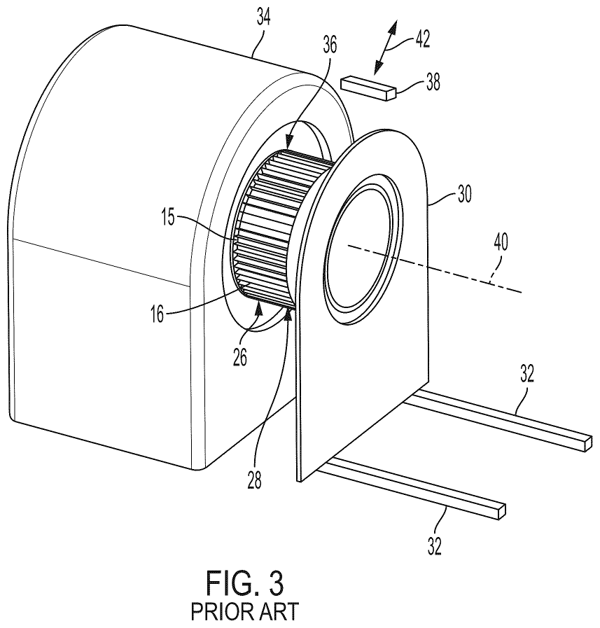

### DETAILED DESCRIPTION

Although various embodiments that incorporate the teachings of the present disclosure have been shown and described in detail herein, those skilled in the art can readily devise many other varied embodiments that still incorporate these teachings. The scope of the disclosure is not limited in its application to the exemplary embodiment details of construction and the arrangement of components set forth in the description or illustrated in the drawings. The disclosure encompasses other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass direct and indirect mountings, connections, supports, and couplings. Further, “connected” and “coupled” are not restricted to physical or mechanical connections or couplings.

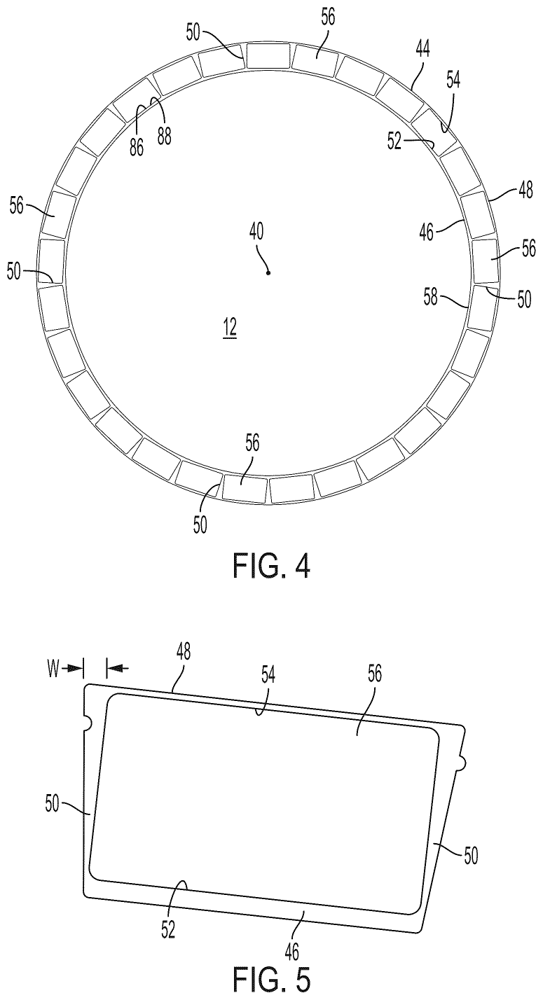

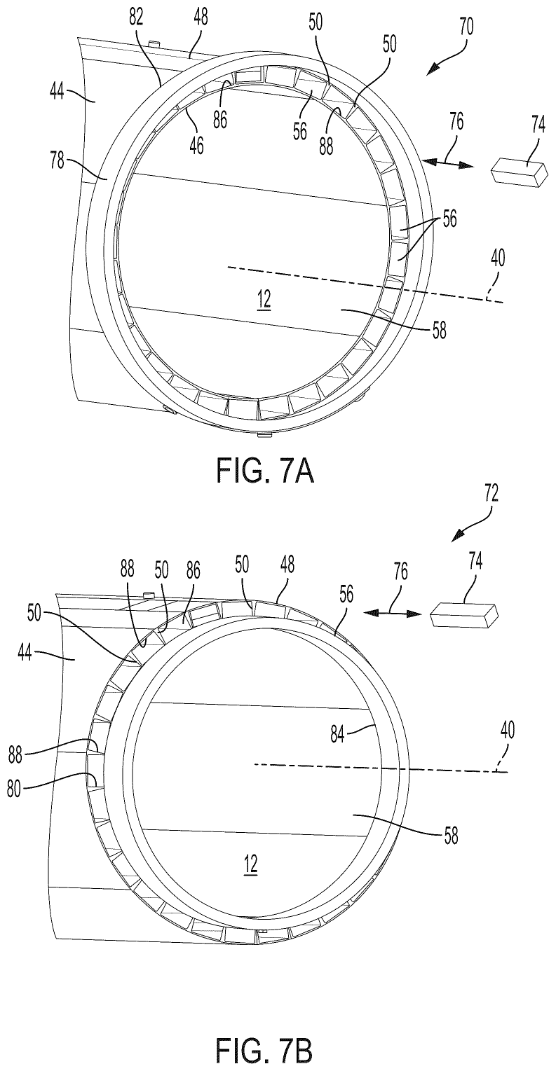

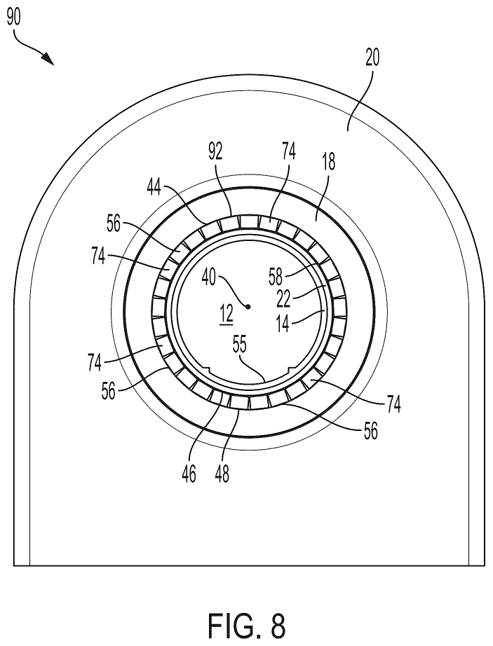

Referring to FIG. 4, a front view of a gantry tube 44 used to form a PET gantry in accordance with an aspect of the invention is shown. The gantry tube 44 is part of an imaging system such as a magnetic resonance/positron emission tomography (MR/PET) imaging system 90 (see FIG. 8). The gantry 44 includes a first tube 46 having an inner surface 58 and an outer surface 52. The first tube 46 is located within a second tube 48. The first tube 46 is positioned about the longitudinal axis 40. The gantry tube 44 further includes a plurality of wall elements 50 connected between the first 46 and second 48 tubes. Each wall 50 extends between the outer surface 52 of the first tube 46 and an inner surface 54 of the second tube 48 to form a plurality of channels 56 between the first 46 and second 48 tubes. In accordance with an aspect of the invention, each channel 56 holds an associated PET detector 74 of the system 90 (FIG. 8). Further, each channel 56 is configured to receive an associated PET detector 74 in an axial direction as will be described in connection with FIGS. 7A-7B.

FIG. 5 depicts an enlarged view of an exemplary channel 56. A size or width W of each wall 50 gradually increases as the wall 50 extends from the first tube outer surface 52 toward the second tube inner surface 54 to form a substantially triangularly or wedge-shaped wall 50. This forms a channel 56 having a substantially rectangular shape suitable for receiving a single PET detector 74. Alternatively, the channel 56 may have other shapes suitable for receiving a corresponding PET detector shape such as square shape, for example.

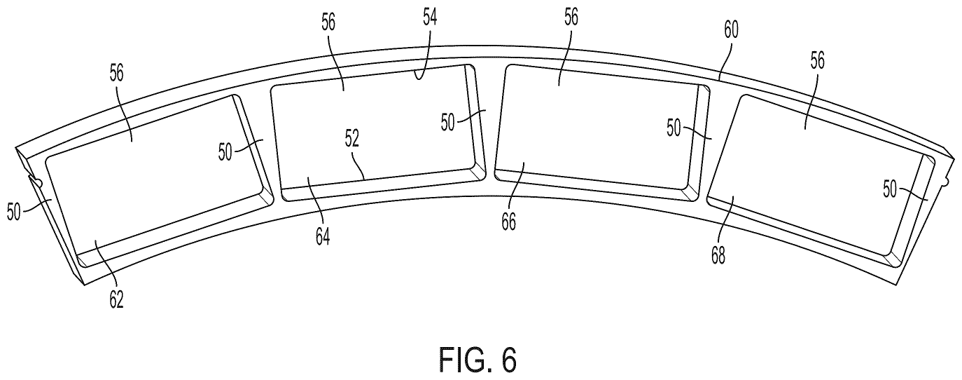

Referring to FIG. 6, a segment 60 of the gantry tube 44 is shown. In an embodiment, the segment 60 has a substantially curved shape and includes first 62, second 64, third 66 and fourth 68 channels. The gantry tube 44 may be formed by joining several segments 60. For example, the segments 60 may be joined on a mandrel using adhesives and mechanical locking features. In aspect of the invention, assembly of the segments 60 on a mandrel provides a uniform and repeatable surface for the RF screen 22 (see FIG. 8). In particular, the RF screen 22 is placed on the mandrel before the segments 60 are bonded together and to the RF screen 22.

The segment 60 may be fabricated using a polymer material and is formed by a pultrusion process that enables formation of thin walls suitable for forming the channels 56. It has been found by the inventors herein that the pultrusion technique may be used to form a segment 60 having a single channel 56 although the formation of additional channels 56, such as three or more channels 56, has been found to provide more suitable results. Further, a segment 60 that includes a channel 56 that is not desired may be removed by machining away the channel 56. For example, a single channel 56 may be machined away from a segment 60 having an odd number of channels 56. In an alternative method of fabrication, a large autoclave is used to join segments 60 that are made from pre-impregnated materials. The segments 60 are then cured under the vacuum and heat of the autoclave to form the gantry tube 44.

Referring to FIGS. 7A and 7B, perspective patient 70 and service 72 end views of the gantry tube 44 are shown. The gantry tube 44 and channels 56 are oriented in a longitudinal direction about the longitudinal axis 40. In accordance with an aspect of the invention, the longitudinal orientation of each channel 56 enables insertion of a PET detector 74 (exemplary PET detector 74 is shown in FIGS. 7A and 7B) in each channel 56, or the removal of a PET detector 74 from a channel 56, in an axial direction (i.e., as shown by arrow 76) substantially parallel to the longitudinal axis 40. The gantry tube 44 is stationary and thus the PET detectors 74 are inserted or removed without moving or extending the gantry tube 44 relative to a remaining portion of the system 90.

A patient is received into the patient bore 12 via the patient end 70 (FIG. 7A). Servicing of the gantry tube 44 may be performed via the service end 72 (FIG. 7B) which is opposite the patient end 70. The channels 56 extend between the patient 70 and service 72 ends. In accordance with an aspect of the invention, each PET detector 74 may be inserted into an associated channel 56 in the axial direction 76 via the patient end 70 or the service end 72.

First 78 and second 80 support rings are located on the patient 70 and service 72 ends, respectively, of the gantry tube 44. The support rings 78, 80 may be removably attached to the gantry tube 44 and serve as an anchor point for the body coil 14 of the MRI portion of the system 90. In an embodiment, the first 78 and second 80 support rings are attached to an outer diameter 82 of the second tube 48 at the patient end 70 and an inner diameter 84 of the first tube 46 at the service end 72, respectively, of the gantry tube 44. This enables mounting of the gantry tube 44 inside the gradient coil 18 while still allowing access to the PET detectors 74 positioned in the channels 56 and shims that are used to adjust a position the gradient coil 18. Further, the support rings 78, 80 provide additional stiffness to the gantry tube 44.

The support rings 78, 80 are located outside of a field of view of the PET detectors 74 such that they do not attenuate a PET signal generated by the PET portion of the system 90. The support rings 78, 80 are optimally placed for mounting the gantry tube 44 to the system 90. In accordance with an aspect of the invention, the body coil 14, a component that is easily damaged in service, may be exchanged without removing the gantry tube 44.

Each segment 60 may be fabricated from a polymer enhanced with glass fiber such as glass reinforced plastic. Alternative materials having slight to moderate electrical conductivity, such as carbon fiber, polymers enhanced with copper or metallic additives and metallic meshes may be used to provide both structural strength and shielding against electromagnetic interference (EMI) generated during operation of the system 90. In particular, a balance must be achieved between providing proper shielding and the effects of eddy current heating on a conductive structure. In accordance with an aspect of the invention, a metallic surface 86 may be formed on an inside surface 88 of at least one channel 56 to form a waveguide. For example, the metallic surface 86 may be a metallic coating that is applied to the inside surface 88 or a metallic foil that is laminated on to the inside surface 88. In another embodiment, each segment 60 is fabricated from a nonconductive material having sufficient structural stability.

It has been found by the inventors herein that the pultrusion process produces segments 60 that are highly stable and precise. The geometry and reproducibility of the inner surface 58 of the first tube 46 is acceptable when a mandrel is used. A shape of an RF screen carrier should be precise and the inner surface of the segment 60 provides a suitable surface for the screen while adding no extra supporting features. Further, gaps between PET detectors 74 are minimized and the polymer profile in front of the PET detectors 74 has low attenuation. For example, the gaps may be approximately 1-3 mm in size.

Referring to FIG. 8, a front view of an MR/PET imaging system 90 is shown that includes the gantry tube 44 of the invention. The system 90 includes the patient bore 12 that receives a patient to be scanned, body coil 14, gantry tube 44 that forms a part of a PET gantry 92, gradient coil 18, superconducting magnet 20 and the RF screen 22 located on the first tube inner surface 58. Further, the patient bore 12 is defined by an inner surface 55 of the body coil 14.

In an aspect of the invention, a gantry tube 44 for a system 90 is disclosed that enables insertion or removal of PET detectors 74 in and out of associated channels 56 of the gantry tube 44 by sliding the PET detectors 74 in and out of the associated channels 56 in an axial direction 76 at either the patient end 70 or service 72 end of the gantry tube 44. Access to the PET detectors 74 is provided without removing the gantry tube 44 from the magnet 20, thus reducing system downtime and the risk of damage to equipment. Since the gantry tube 44 does not move and remains inside the gradient coil 18, only a simple attachment to the magnet 20 is needed thus simplifying the mechanical structure of the system 90 (i.e., no rails, bearing, backplanes, etc. are needed). The risk of injury from moving a heavy gantry is substantially reduced and specialty tools are not required for performing service. In addition, the invention provides a known and reproducible surface to attach an RF shield of the body coil 14. Further, the RF cabin (i.e., a scan room wherein system 90 is located) may be optimized and reduced in size since the gantry tube 44 does not extend from the system 90 and thus an extended PET gantry length does not factor into room size requirements. In addition, the material used to fabricate the gantry tube 44 has low attenuation for the PET signal and gantry fabrication is very precise allowing for the accurate location of each PET detector 74 in the gantry tube 44.

In an alternative embodiment, the PET gantry 92 including gantry tube 44 may be integrated directly into the gradient coil 18. In this embodiment, the effects of vibration and heat on the PET detectors 74 should be minimized since both are detrimental to the associated electronics and operation of the PET detector 74.

Another embodiment would convolve the body coil 14 and PET gantry 92. In this embodiment, the PET detectors 74 reside within a radio frequency alternating field (B1) reflow space generated by the system 90 and would require significantly higher levels of EMI shielding. In addition, associated cabling may cause an antenna effect (due to currents on the cable shields). Further, shielded structures in this volume would be parasitic and require more power to offset the effect.

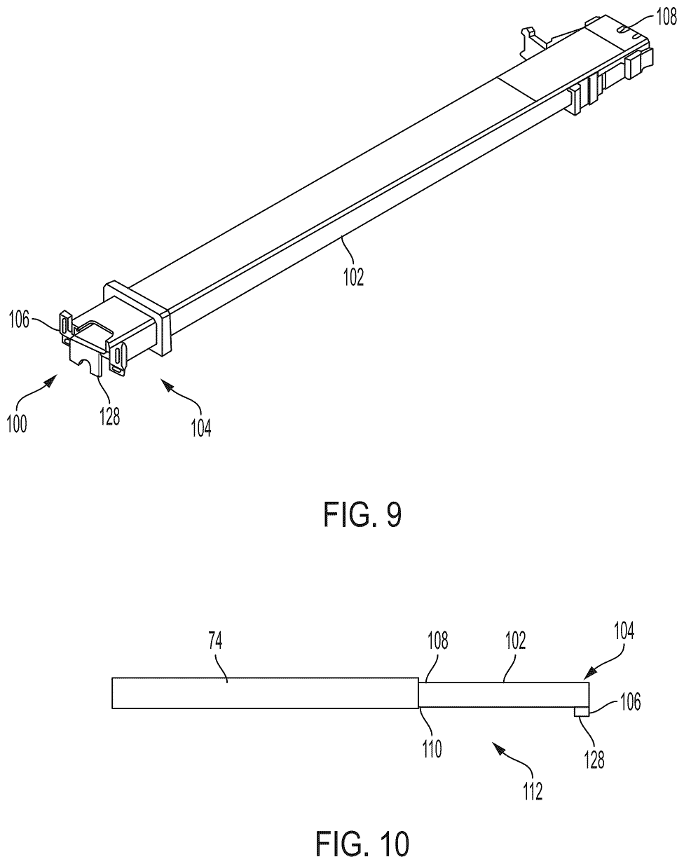

Referring to FIGS. 9-12, an embodiment for a PET detector positioning device 100 for positioning an associated PET detector 74 in a respective channel 56 is shown. Referring to FIG. 9, the positioning device 100 includes an extension tube 102 that is associated with each PET detector 74. A first end 104 of the extension tube 102 includes a rotatable latch device 106. Referring to FIG. 10, a second end 108 of the extension tube 102 is attached to an end 110 of a PET detector 74 to form a tube and PET detector assembly 112.

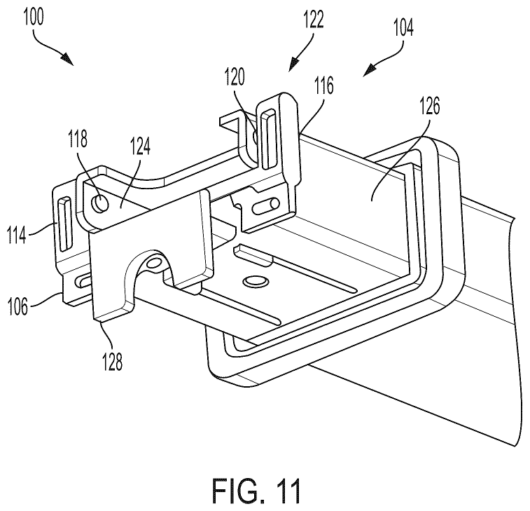

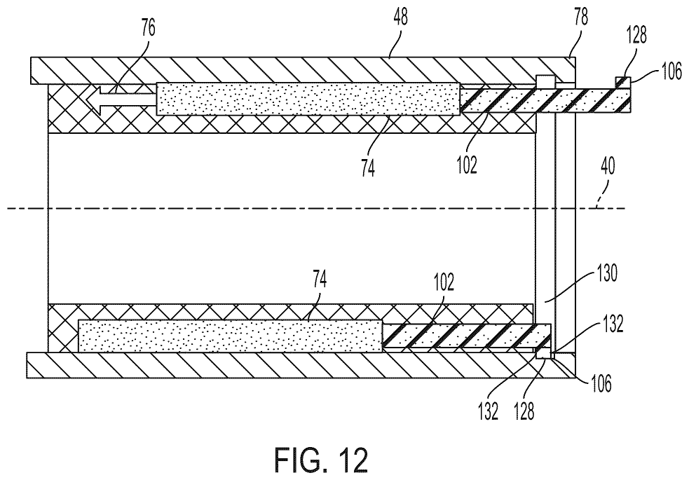

Referring to FIG. 11, a perspective view of the latch device 106 is shown. The latch device 106 includes first 114 and second 116 arms having first 118 and second 120 pivot elements that form a bailer arm 122. The first 118 and second 120 pivot elements extend through first 124 and second 126 side surfaces of the extension tube 102, respectively, to enable rotation of the bailer arm 122 relative to the extension tube 102. The latch device 106 further includes a projecting latch element 128 that extends away from the longitudinal axis 40 (see FIGS. 7A and 12) when the latch element 128 is rotated into a detent of the latch device 106 and into a locked or secured position.

Referring to FIG. 12, a partial side view of the first support ring 78 and second tube 48 is shown. The first support ring 78 includes an annular groove 130 sized to receive the latch element 128 and defined by side walls 132. The annular groove 130 serves as an axial alignment notch 134 used to set or locate an axial position of an associated PET detector 74. During assembly, the tube and PET detector assembly 112 is moved in the axial direction 76 and inserted into an associated channel 56. Upon alignment of the latch element 128 with the annular groove 130, the bailer arm 122 is rotated toward the annular groove 130. This rotates the latch element 128 into the locked position and inserts the latch element 128 in the annular groove 130. When this occurs, a precise distance is set between the axial alignment notch 134 and the PET detector 74. The PET detector 74 may be a known silicon photomultiplier (SiPM) based detector having a plurality of mini-blocks of pixels wherein each mini-block includes an array of pixels. In an embodiment, the distance may be set between the axial alignment notch 134 and a known first crystal of the PET detector 74, i.e., a row of pixels of the first mini-blocks of pixels closest to where the patient enters the patient bore 12 for a scan. In an embodiment, the first crystal marks the position of the PET detector 74 within the PET gantry 92. In addition, the latch element 128 is captured between the side walls 132 of the annular groove 130 which inhibits movement of the PET detector 74. In order to remove the tube and PET detector assembly 112 from an associated channel 56, the bailer arm 122 is rotated away from the annular groove 130, thus disengaging the latch element 128 an enabling axial movement to enable removal of the tube and PET detector assembly 112.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

We claim:

1. A gantry tube for a magnetic resonance/positron emission tomography (MR/PET) imaging system that includes a gradient coil located within a superconducting magnet and wherein the imaging system further includes a body coil located within a radio frequency (RF) screen and wherein the gantry tube is located between the gradient coil and RF screen, comprising:
a first tube located within a second tube, wherein the first tube is oriented about a longitudinal axis of the imaging system and wherein an inner surface of the body coil defines a patient bore for receiving a patient to be scanned; and
a plurality of wall elements that extend between the first and second tubes, wherein the walls and first and second tubes form a plurality of channels that extend in an axial direction substantially parallel to the longitudinal axis wherein each channel is configured to hold a PET detector of the imaging system and wherein a PET detector is inserted into or removed from an associated channel in the axial direction and wherein the gantry tube is fabricated by joining a plurality of segments that each include sections of the first and second tubes, walls and a plurality of channels and wherein each segment is fabricated using a nonconductive material.

2. The gantry tube according to claim 1, further including first and second support rings located at first and second ends, respectively, of the gantry tube to provide additional stiffness to the gantry tube.

3. The gantry tube according to claim 1, wherein a PET detector is inserted into or removed from an associated channel without moving the gantry tube relative to the imaging system.

4. The gantry tube according to claim 1, wherein each segment includes first, second, third and fourth channels.

5. The gantry tube according to claim 1, wherein the segments are formed by a pultrusion process.

6. The gantry tube according to claim 1, wherein a width of each wall element increases as the wall extends from the first tube to the second tube to form a substantially wedge-shaped wall.

7. A gantry tube for a magnetic resonance/positron emission tomography (MR/PET) imaging system that includes a gradient coil located within a superconducting magnet and wherein the imaging system further includes a body coil located within a radio frequency (RF) screen, comprising:
a first tube located within a second tube, wherein the first tube is oriented about a longitudinal axis of the imaging system; and
a plurality of wall elements that extend between the first and second tubes, wherein a width of each wall element increases as the wall extends from the first tube to the second tube to form a substantially wedge-shaped wall and wherein the walls and first and second tubes form a plurality of channels that extend in an axial direction substantially parallel to the longitudinal axis wherein each channel is configured to hold a PET detector of the imaging system and wherein a PET detector is inserted into or removed from an associated channel in the axial direction from either a first end or a second end of the gantry tube and wherein the gantry tube is fabricated by joining a plurality of segments that each include sections of the first and second tubes, walls and a plurality of channels and wherein each segment is fabricated using a nonconductive material.

8. The gantry tube according to claim 7, further including first and second support rings located at first and second ends, respectively, of the gantry tube to provide additional stiffness to the gantry tube.

9. The gantry tube according to claim 7, wherein a PET detector is inserted into or removed from an associated channel without moving the gantry tube relative to the imaging system.

10. The gantry tube according to claim 7, wherein each segment includes first, second, third and fourth channels.

11. The gantry tube according to claim 7, wherein the segments are formed by a pultrusion process.

12. The gantry tube according to claim 7, wherein each channel has a substantially rectangular shape.

13. The gantry tube according to claim 7, wherein at least one channel includes a metallic surface that forms a waveguide.

14. A method of inserting a PET detector into a gantry tube of a magnetic resonance/positron emission tomography (MR/PET) imaging system or removing a PET detector from the gantry tube wherein the imaging system includes a gradient coil located within a superconducting magnet and the imaging system further includes a body coil located within a radio frequency (RF) screen and wherein the gantry tube is located between the gradient coil and the RF screen, comprising:
providing a first tube located within a second tube, wherein the first tube is oriented about a longitudinal axis of the imaging system;
providing a plurality of wall elements that extend between the first and second tubes to form a plurality of channels wherein the channels extend in an axial direction substantially parallel to the longitudinal axis;
joining a plurality of segments that each include sections of the first and second tubes, walls and a plurality of channels to form the gantry tube wherein each segment is fabricated using a nonconductive material; and
moving a PET detector in the axial direction to either insert the PET detector into an associated channel or remove a PET detector from an associated channel.

15. The method according to claim 14, further including providing first and second support rings located at first and second ends, respectively, of the gantry tube to provide additional stiffness to the gantry tube.

16. The method according to claim 14, wherein a PET detector is inserted into or removed from an associated channel without moving the gantry tube relative to the imaging system.

17. The method according to claim 14, wherein each segment includes first, second, third and fourth channels.

18. The method according to claim 14, wherein the segments are formed by a pultrusion process.

19. The method according to claim 16, further including increasing a width of each wall element as the wall extends from the first tube to the second tube to form a substantially wedge-shaped wall.

20. The method according to claim 19, wherein each channel has a substantially rectangular shape.

21. A PET detector positioning device for a magnetic resonance/positron emission tomography (MR/PET) imaging system that includes a gradient coil located within a superconducting magnet and wherein the imaging system further includes a body coil located within a radio frequency (RF) screen, wherein the imaging system also includes a PET gantry having a plurality of channels that receive respective PET detectors wherein the channels extend in an axial direction substantially parallel to a longitudinal axis of the imaging system, comprising:
a support ring that supports the PET gantry, wherein the support ring includes an annular groove that forms an axial alignment notch;
an extension tube having first and second ends wherein the second end of the extension tube is attached to a first end of an associated PET detector;
a bailer arm rotatably attached to the first end of the extension tube, wherein the bailer arm includes a latch element that engages the annular groove to set a desired distance between the axial alignment notch and the PET detector.

22. The PET positioning device according to claim 21, wherein the extension tube and PET detector are inserted in the axial direction into an associated channel and the latch element engages the annular groove when the latch element and annular groove are aligned.

23. The PET positioning device according to claim 21, wherein the bailer arm includes first and second pivot elements that extend through first and second surfaces of the extension tube to enable rotation of the bailer arm relative to the extension tube.

24. The PET positioning device according to claim 21, wherein a desired distance is set between the axial alignment notch and a section of the PET detector that marks the position of the PET detector within the gantry.

25. The PET positioning device according to claim 24, wherein the desired distance is set between the axial alignment notch and a first crystal of the PET detector.
